(19) United States
(12) Patent Application Publication
(10) Pub. No.: US 2008/0109037 A1
(43) Pub. Date: May 8, 2008

(54) Press fit suture anchor and inserter assembly

(75) Inventors: Anton J. Steiner, Wharton, NJ (US); David I. Knight, Hopelawn, NJ (US); Cesar D. Chavista Para, Jersey City, NJ (US); James Shock, Glen Rock, NJ (US)

Correspondence Address: JOHN S. HALE;GIPPLE & HALE, 6665-A OLD DOMINION DRIVE, MCLEAN, VA, 22101, US

(73) Assignee: Musculoskeletal Transplant Foundation

(21) Appl. No.: 11/592,183

(22) Filed: Nov. 3, 2006

(51) Int. Cl. A61B 17/04

(52) U.S. Cl. 606/232

(57) ABSTRACT

A sterile suture anchor and insertion kit with the suture anchor having a cylindrical body portion with a generally tapered distal end portion and flat end. A plurality of parallel longitudinal grooves are cut into the cylindrical body and a connecting throughgoing bore is cut through the cylindrical body engaging the plurality of longitudinal grooves. An inserter driver is adapted to be mounted to the suture anchor, the driver comprising a handle with a drive shaft mounted to the handle, and a slidable sleeve mounted on the drive shaft which holds the suture anchor. A guide member comprising a handle with a hollow sleeve extending therefrom is used to hold a punch against the area on which a blind bore is formed to form the blind bore after which the inserter driver is mounted in the guide member sleeve to guide the slidable sleeve of the inserter driver and associated suture anchor into the blind bore.

### RELATED APPLICATIONS

This are no related applications.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

The field of art to which this invention relates is generally directed to suture anchors and more specifically to a press fit suture anchor constructed of allograft bone which holds a suture and a punch and inserter device for inserting the suture anchor.

2. Description of the Prior Art

As the treatment of injuries to joints and soft tissue has progressed, a need has developed for medical devices which can be used to attach tendons, ligaments and other soft tissue to bone. When surgically repairing an injured joint, it is preferable to restore the joint by reattaching the damaged soft tissues such as ligaments and tendons to a bone rather than replacing them with an artificial material.

An increase in the incidence of injuries to joints involving soft tissue has been observed. This increased incidence of injuries may be due, at least in part, to an increase in participation by the public in various physical activities such as sports and other recreational activities. These types of activities increase the loads and stress placed upon joints, sometimes resulting in joint injuries with corresponding damage to associated soft tissue. There are well over 500,000 surgical procedures performed in the United States annually in which soft tissue was attached to a bone in various joints including the shoulder, hip and knee.

One conventional orthopedic procedure for reattaching soft tissue to bone is performed by initially drilling holes or tunnels at predetermined locations through a bone in the vicinity of a joint. The surgeon approximates soft tissue to the surface of the bone using sutures threaded through these holes or tunnels. This method is a time consuming procedure resulting in the generation of numerous bone tunnels. The bone tunnels, which are open to various body fluids and infectious agents, may become infected, resulting in bone breakage and complications such as a longer bone-healing period may result. A known complication of drilling tunnels across bone is that nerves and other soft tissue may be injured by the drill bit or orthopedic pin as it exits the far side of the bone. Also, it may be anatomically impossible or at least very difficult to reach and/or secure a suture that has been passed through a tunnel. When securing the suture or wire on the far side of the bone, nerves and soft tissues can also become entrapped and damaged.

Screws are also used to secure soft tissues adjacent to the bone surface. Screws suffer from a disadvantage in that they tend to loosen with time, thereby requiring a second operation to remove the loosened screw. In addition, when the screws are set in bone, the heads of the screws frequently protrude above the surface of the bone in which they are set, thereby presenting an abrasive surface which may create wear problems with surrounding tissue. Once a hole has been made in the bone it may be impossible to relocate the hole a small distance away from its original position due to the disruption of the bone structure created by the initial hole. Finally, the nature of a screw attachment tends to require a flat attachment geometry as the pilot hole must generally be located on a relatively flat section of the bone, and toothed washers must frequently be used in conjunction with the screws to fasten the desired objects to the target bone. As a result of these constraints, it may be necessary to locate the attachment point at less than an optimal position.

Staples are also used to secure soft tissue adjacent the bone surface. Staples frequently have to be removed after they have been in position for some time, thereby necessitating a second operation. In addition, staples must generally be positioned so as to maximize their holding power in the bone which may conflict with the otherwise-optimal position for attachment of the objects to bone. Staples have also been known to crack the bone during deployment, or to accidentally transect the object (e.g. soft tissue) being attached to the bone, since it tends to be difficult to precisely control the extent of the staple's penetration into the bone. Additionally, once the staple has been set into the bone, the position of the staple is then effectively determined, thereby making it impossible to thereafter adjust the position of the staple or to adjust the degree of tension being applied to the object which is being attached to the bone without removing the staple and setting a new staple.

In order to overcome a number of the problems associated with the use of the conventional soft tissue to bone attachment procedures, suture anchors have been developed and are now frequently used to attach soft tissue to bone. A suture anchor, commonly referred to as a bone anchor, is an orthopedic, medical device which is typically implanted into a cavity drilled or punched into a bone. The bone cavity is generally referred to as a bore hole and if it does not extend through the bone is typically referred to as a “blind hole”. The bore hole is typically drilled through the outer cortical layer of the bone and into the inner cancellous layer. The suture anchor may be engaged in the bore hole by a variety of mechanisms including friction fit, barbs which are forced into the cancellous layer of bone or by threading into pre-threaded bores in the bone mass or using self tapping threads. Suture anchors have many advantages including reduced bone trauma, simplified application procedures, and decreased likelihood of suture failure. Suture anchors may be used in shoulder reconstruction for repairing the glenohumeral ligament and may also be used in surgical procedures involving rotator cuff repair, ankle and wrist repair, bladder neck suspension, and hip replacement.

Suture anchors typically have a hole or opening for receiving a suture. The suture extends out from the bore hole and is used to attach soft tissue. The suture anchors presently described in the art may be made of absorbable materials which absorb over time, or they may be made from various non-absorbable, biocompatible materials. Although most suture anchors described in the art are made from non-absorbable materials, the use of absorbable suture anchors may result in fewer complications since the suture anchor is absorbed and replaced by bone over time. The use of absorbable suture anchors may also reduce the likelihood of damage to local joints caused by anchor migration. Moreover, when an absorbable suture anchor is fully absorbed it will no longer be present as a foreign body. It is also advantageous to construct the bone anchor out of allograft cortical bone as this material will result in natural filling in of the bore with bone in the original bone base and the elimination of foreign material from the site. Another problem in the prior art is that the suture does not glide easily through the anchor making typin knots and sliding the knot to secure the tissue difficult.

It is also a problem that most of the bone anchors currently used are prepacked with sutures attached in kit form forcing the surgeon to use a specific type of suture and the hospital to carry large numbers of bone anchors in inventory with varying suture sizes.

A number of prior art patents such as U.S. Pat. Nos. 6,508,830; 5,941,882 and 5,733,307 are directed toward threaded bone anchors which have driver positioning grooves or troughs cut longitudinally along the anchor body intersecting the threads to receive sutures during the bone anchor insertion process and to receive an associated driver. The U.S. Pat. No. 6,508,830 patent discloses a smooth surfaced suture anchor with a flat end surface defining an angled cut leading to a transverse suture throughgoing bore cut in the anchor body.

U.S. Pat. No. 5,824,011 is directed toward a threaded bone anchor with a suture receiving eyelet. The anchor body has channels cut into its sides to receive driver torque applicators. The anchor is provided with a male member having a suture receiving eyelet, the male member fitting into a same shaped female configuration in the driver head.

U.S. Pat. No. 6,111,164 shows a bone insert which is formed from human cortical bone which is adapted to be driven into bone and the aforementioned U.S. Pat. No. 6,508,830 shows a threaded allograft bone anchor which can be mounted into the bone.

Although suture anchors for attaching soft tissue to bone are available for use by the orthopedic surgeon, there is a need in this art for novel suture anchors having improved performance characteristics, such as ease of insertion and greater resistance to “pull-out”.

### SUMMARY OF THE INVENTION

The present invention is directed toward a suture anchor constructed of allograft human bone which is press fit into a bone bore hole and has a smooth outer surface with plurality of longitudinal grooves cut into its outer surface to hold a suture loop.

The present invention provides a technical advantage in that it provides a channel in the suture anchor in which a suture loop resides during insertion of the bone anchor into the bone while also allowing the driver to apply a driving force to the proximal end of the anchor so that the anchor is less susceptible to mechanical breakage.

Accordingly, one of the objects of the present invention is to provide an allograft suture anchor which promotes the use of natural bone growth in the bone bore hole.

An additional advantage is a suture anchor made of allograft bone is radiopaque for the time it takes to incorporate making it visible under x-ray imaging.

It is another object of the present invention to provide a suture anchor in which the suture glides easily through the anchor to facilitate typing knots and sliding to knot to secure the tissue.

It is still another object of the present invention to provide a suture anchor which can be used with a wide variety of sutures from different manufacturers allowing the surgeon the choice of sutures and suture composition.

It is another object of the present invention to provide a suture anchor which is simple to apply and is mechanically stable when implanted in bone.

It is a further object of the present invention to provide an absorbable suture anchor made of cortical bone.

It is still another object of the present invention to provide a novel suture anchor for anchoring one end of a piece of conventional suture in bone which has high tissue acceptability, prevents back out and is reliable in use.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 is a perspective view of the inventive bone suture anchor;

FIG. 2 is an enlarged side elevational view of the suture anchor shown in FIG. 1;

FIG. 3 is a rear elevational view of the suture anchor shown in FIG. 2;

FIG. 4 is a side elevational view of the suture anchor of FIGS. 1-3 mounted in a delivery device;

FIG. 5 is an enlarged partial side elevational view of a section of the insert device anchor sleeve, punch and suture anchor shown in FIG. 4 showing the anchor before extension;

FIG. 6 is an enlarged partial side elevational view of a section of the insert device anchor sleeve, punch and suture anchor shown in FIG. 4 showing the anchor after extension;

FIG. 7 is a side elevational view of a punch and inserter guide and holder used with the invention;

FIG. 8 is an enlarged front elevational view of the guide sleeve of the punch and inserter guide and holder shown in FIG. 7;

FIG. 9 is a front elevational view of the guide sleeve of the punch and inserter guide and holder of FIG. 8;

FIG. 10 is an enlarged side elevation view of the handle of the punch and inserter guide and holder shown in FIG. 7;

FIG. 11 is a cross section taken along line 11′-11′ of the handle of FIG. 10;

FIG. 12 is a top plan view of the handle shown in FIG. 10;

FIG. 13 is a side elevational view of the punch device;

FIG. 14 is an enlarged front elevational view of the tip of the punch device of FIG. 13 taken from the direction shown by line 14′-14′;

FIG. 15 is a perspective view of the punch mounted in the punch and inserter guide and holder prior to being driven into a bone which is shown in cross section;

FIG. 16 is an enlarged view taken from circle A of FIG. 15 showing the punch prior to entry into the bone;

FIG. 17 is a perspective view of the punch mounted in the punch and inserter guide and holder after being driven into a bone which is shown in cross section;

FIG. 18 is an enlarged view taken from circle B of FIG. 17 showing the punch driven into the bone to form the blind bore;

FIG. 19 is a perspective view of the anchor inserter device mounted in the punch and inserter guide and holder prior to the suture anchor being driven into a bone bore;

FIG. 20 is an enlarged view taken from circle C of FIG. 19 showing the suture anchor prior to entry into the bone bore;

FIG. 21 is a perspective view of the anchor inserter device mounted in the punch and inserter guide and holder driving the suture anchor into the bone bore;

FIG. 22 is an enlarged view taken from circle D of FIG. 21 showing the suture anchor driven into the bone bore;

FIG. 23 is a perspective view of the suture anchor mounted in the bone bore with suture strands mounted thereto after removal of the anchor inserter device; and

FIG. 24 is an enlarged view taken from circle E of FIG. 23 showing the suture anchor in the bone bore with the suture extending therefrom through the punch and inserter guide and holder sleeve.

### DETAILED DESCRIPTION OF THE PREFERRED EMBODIMENT

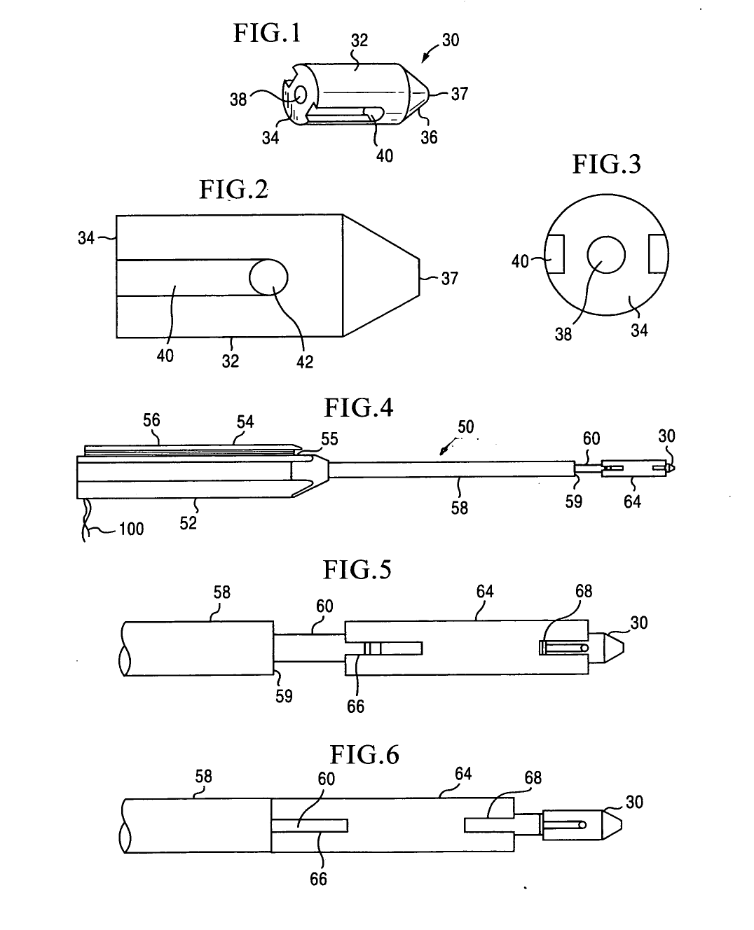

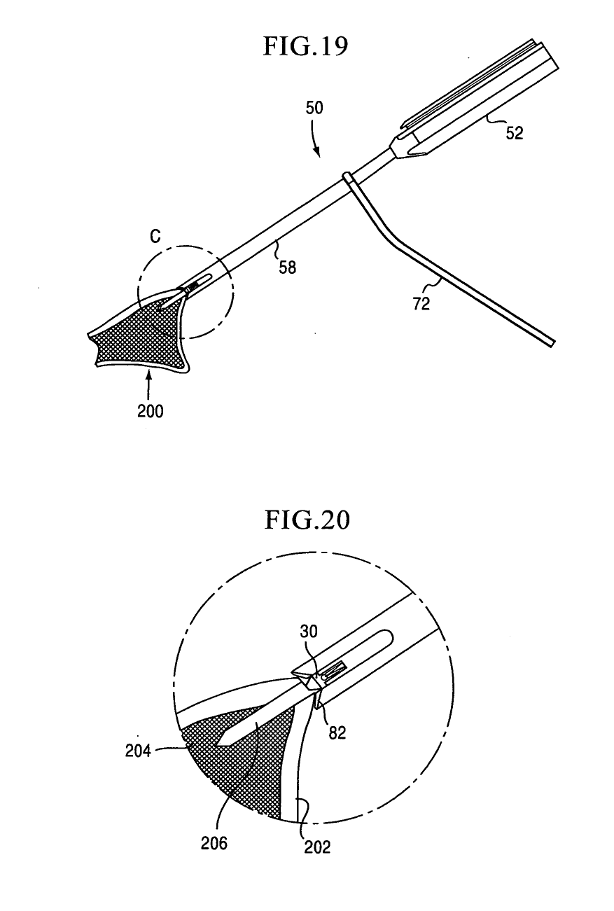

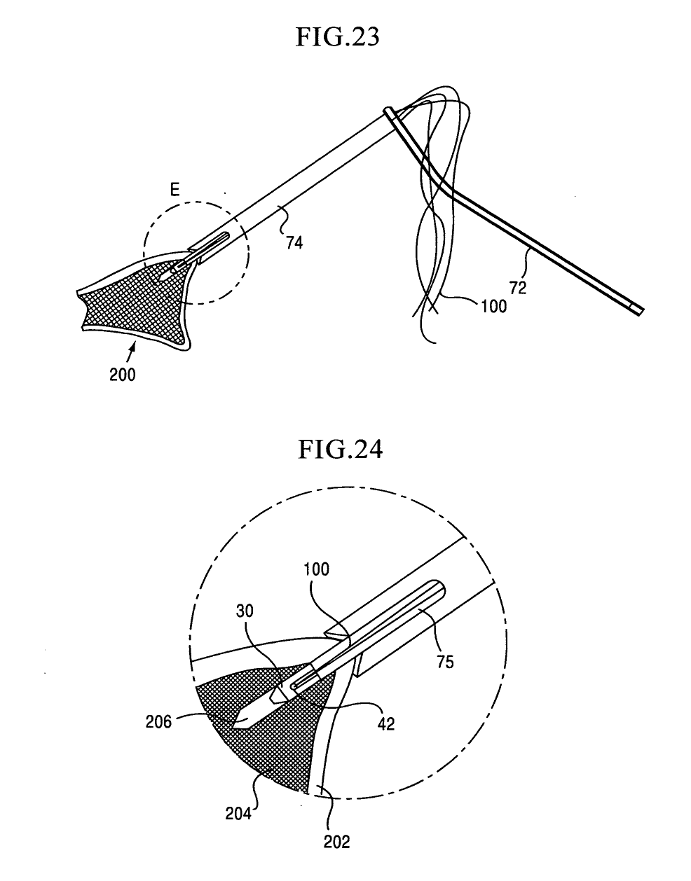

The preferred embodiment and the best mode of the invention as shown in FIGS. 1 through 24 shows a suture anchor 30 with a cylindrical body 32 having a flat proximal end 34 and a truncated conical distal portion 36 having a flat end surface 37 which is initially inserted into a bore 206 cut in the bone mass as shown in FIGS. 19-24. The flat proximal end 34 defines a centrally located blind bore 38 which is sized to receive the end 62 of an inserter device 50. The conical distal end 37 tapers inward in about from 30° to 45° from the sides of the cylindrical body toward the center longitudinal axis of the suture anchor for self centering insertion into bone bore 206. Side grooves 40 are cut in the exterior surface of the cylindrical body and run from about 50% to about 75% of the length of the cylindrical body 32 where they intersect a transverse throughgoing bore 42 in the cylindrical body 32 and together form a seat in the suture anchor for the suture strand or strands 100 which are most clearly shown in FIGS. 22-24. Preferably, the suture anchor 30 is manufactured from human allograft bone which is formed of mineralized cortical bone which has been freeze dried to reduce its water content. Alternatively the anchor body may be partially demineralized and alternately treated with bone morphogenic protein, hylauronic acid and a phosphate buffer for quicker bone formation once the suture anchor has been threaded into the bone.

It is also envisioned that the suture anchor may be manufactured from a biocompatible and bioresorbable material such as xenograft bone, plastic or a biocompatible metal such as titanium or stainless steel.

The proximal end 34 of the suture anchor 30 is flat for minimum soft tissue impingement after insertion of the suture anchor 30 in the bone. The width of each groove 40 is approximately 1.25 mm±0.05 mm and each groove 40 is positioned on opposite sides of the anchor body. The diameter of the cylindrical body 32 suture anchor 30 preferably runs between 4.7 mm and 6.5 mm and has a length ranging from 8.0 mm to 15.0 mm with a preferred length of 8 mm. The channels or grooves 40 have a width greater than or equal to the diameter of the strand of suture 100 and a depth which is preferably at least twice the diameter of the strand of suture loop 100 extending into the anchor body. The suture strand 100 is preferably a #2 suture and a standard suture made of absorbable, synthetic absorbable or non-absorbable material. It is envisioned that the width and depth of grooves 40 can be varied to receive greater size suture strands or a plurality of sutures.

Sutures 100 such as ORTHOBRAID® are initially inserted through the transverse throughgoing bore 42 with the strands tracking in the grooves 40. The suture anchor 30 is adapted for insertion into the distal end of a driver inserter 50 which is shown in FIGS. 4 through 6. The driver inserter 50 has a handle 52 constructed of plastic with one section of the handle forming a suture holder 54. This section defines a groove 55 around which the suture 100 can be wrapped and also defines upper suture guide groove 56. A stainless steel shaft 58 is secured to the handle 52 in a boss (not shown) and the distal end of shaft 58 is stepped at 59 to form a smaller diameter shaft portion 60 with a punch end which is sized to fit into blind bore 38 formed in the flat proximal end of suture anchor 30. A slidable sleeve member 64 is mounted over stepped shaft portion 60 and abuts the step 59 when the anchor 30 is inserted in the blind bore and is separated from the shaft step 59 covering the cylindrical anchor body 32 prior to insertion as seen in FIG. 5 to hold the bone anchor 30 securely within the sleeve. The sleeve member 64 defines a proximal viewing slot 66 and a distal viewing slot 68. The sleeve member 64 is preferably constructed of a medical grade plastic such as Lexan®.

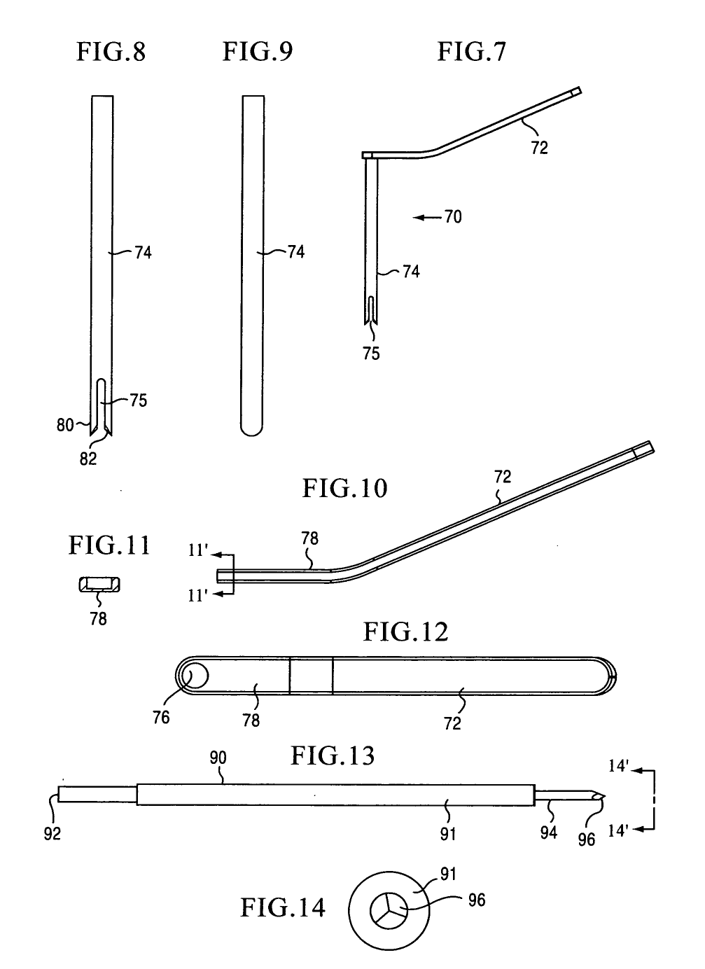

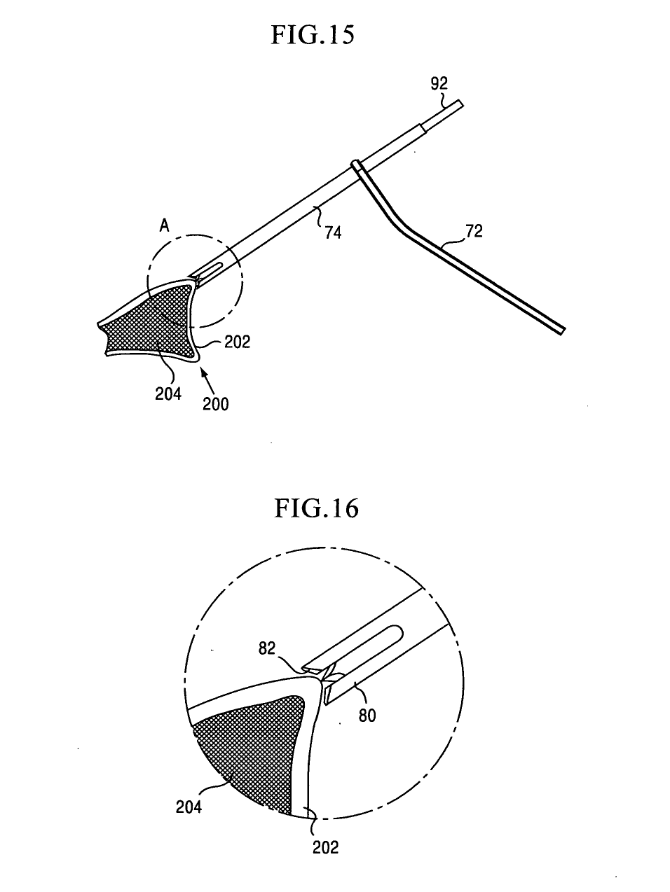

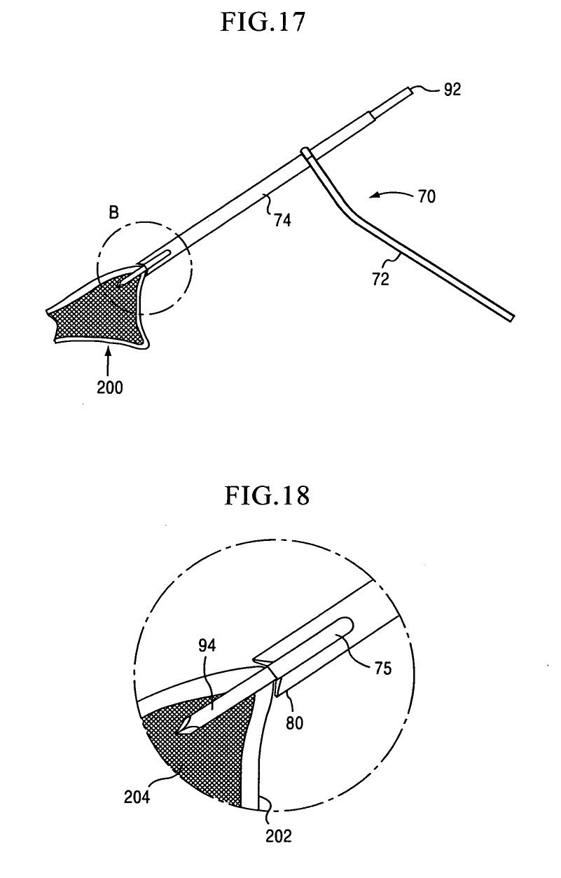

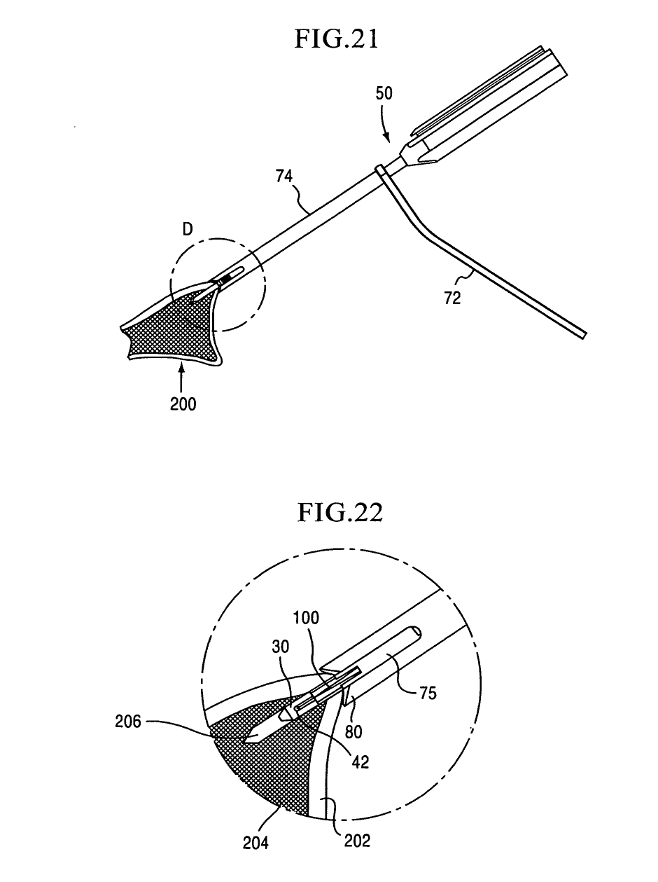

In operation, a combined punch, driver holder 70 as shown in FIGS. 7-12 is placed against the surface of the bone 200 as shown in FIGS. 15 and 16. The punch, driver holder 70 is constructed with an angled handle 72 and a guide sleeve 74 secured in an aperture 76 cut in handle portion 78 of handle 72. The guide sleeve 74 is hollow and formed with opposing side slits 75 at the distal end to allow the user to view the engagement of the punch 90 or the anchor 30 with the bone 200 or bore 206. The tip 80 of the guide sleeve 74 is formed with an end “V” cut 82 to allow the end of the guide sleeve to be easily seated on the bone. Once the guide sleeve 74 is seated on the bone 200 which is shown as a cortical bone layer 202 covering a cancellous bone mass 204, a punch 90 preferably of 3.3 mm diameter having a main shaft 91 as shown in FIGS. 13 and 14 is inserted into the guide sleeve 74 with the proximal portion 92 extending therefrom and is struck by a hammer to drive the distal portion 94. The distal portion 94 has a triangular sharpened point 96 as shown in FIG. 14 and is driven into the bone as shown in FIGS. 17 and 18 to form a bore 206 through the cortical bone layer 202 into the cancellous bone mass 204. The punch 90 is removed and the driver inserter 50 with the attached anchor 30 is inserted into the guide sleeve 74 adjacent the entrance of bore 206 as shown in FIGS. 19 and 20. The end of the driver inserter 50 is struck and the suture anchor 30 is extended as shown in FIG. 6 with the suture anchor 30 being deposited in the bore 206 past the cortical bone layer 202 into the cancellous bone mass 204 as seen in FIGS. 21 and 22.

As the suture anchor 30 is driven into the bone bore 206, the bone mass surrounds the grooves 40 to hold the suture strand(s) 100 within the respective grooves 40 around the bone anchor 30. The suture anchor 30 is then seated in the bore 206 previously formed into the cancellous bone mass 204 with the proximal end past the cortical bone layer 202, the driver inserter 50 having been backed off. Because the suture loop 100 is a single or double strand of material, the failure strength is the suture line break strength rather than the pull out strength of the anchor from the bone. Pull out of the anchor is also diminished because of the deeper seating of the bone anchor and encompassing bone mass as well as a combination of the press fit of the anchor in the bone bore hole and the swelling of the anchor upon hydration at the site since it is freeze dried.

In the foregoing description, the invention has been described with reference to a particular preferred embodiment, although it is to be understood that specific details as shown are merely illustrative, and the invention may be carried out in other ways without departing from the true spirit and scope of the following claims.

1. A sterile suture anchor comprising:
a smooth surfaced substantially cylindrical body with a plurality of parallel longitudinal grooves cut into said cylindrical body;
a throughgoing bore is transversely cut through said cylindrical body with each end of said bore terminating in said longitudinal grooves, said longitudinal grooves and said throughgoing bore being dimensioned to hold at least one suture strand; and
said anchor having a flat proximal end and a cone shaped distal end.

2. A sterile suture anchor according to claim 1 wherein said flat proximal end defines a central blind bore dimensioned to receive the tip of an inserter driver.

3. A sterile suture anchor according to claim 1 wherein said cone shaped distal end terminates in a flat surface forming a truncated cone.

4. A sterile suture anchor according to claim 1 wherein said cone shaped end forms an angle ranging from 30° to 45°.

5. A sterile suture anchor as claimed in claim 1 wherein said suture anchor is constructed of allograft bone.

6. A sterile suture anchor as claimed in claim 1 wherein said suture anchor is constructed of xenograft bone.

7. A sterile suture anchor as claimed in claim 1 wherein said suture anchor is constructed of polymer.

8. A sterile suture anchor as claimed in claim 1 wherein said suture anchor is constructed of a bio-compatible metal.

9. A sterile biocompatible absorbable suture anchor comprising:
a substantially cylindrical body of allograft bone with a tapered distal end portion,
and a flat proximal end, said flat proximal end defining a centrally placed depression therein,
a plurality of parallel longitudinal grooves cut into said cylindrical body;
a throughgoing bore is positioned transverse to a central axis of said cylindrical body and opens into at least two of said longitudinal grooves.

10. A sterile suture anchor according to claim 9 wherein said cylindrical body tapered distal is a truncated cone having an angle ranging from 30° to 45°.

11. A sterile suture anchor as claimed in claim 9 wherein said allograft bone is human cortical bone.

12. A sterile suture anchor and insertion kit comprising:
a suture anchor having a substantially cylindrical body of allograft bone with a tapered distal end portion, and a flat proximal end, said flat proximal end defining a centrally placed depression therein,
a plurality of parallel longitudinal grooves cut into said cylindrical body;
a throughgoing bore is cut in said cylindrical body opening into said plurality of longitudinal grooves; and
a inserter driver adapted to be mounted to said suture anchor, said inserter driver comprising a handle and a shaft mounted to said handle, said shaft being provided a slideable sleeve member adapted to be inserted over said suture anchor.

13. A sterile suture anchor and inserter kit as claimed in claim 12 wherein said inserter driver handle defines a suture holding recess.

14. A sterile suture anchor and inserter kit as claimed in claim 12 wherein said inserter driver handle defines a suture guide channel.

15. A sterile suture anchor and inserter kit as claimed in claim 12 wherein said inserter kit includes a guide member, said guide member comprising a handle and a guide sleeve mounted to said handle.

16. A sterile suture anchor and inserter kit as claimed in claim 15 wherein said guide sleeve is provided with at least one slit running longitudinally along the guide sleeve.

17. A sterile suture anchor and inserter kit as claimed in claim 15 wherein said guide sleeve defines a “V” shaped distal tip.

18. A sterile suture anchor and inserter kit as claimed in claim 15 wherein said handle is angled from a central axis of said guide sleeve.

19. A sterile suture anchor and inserter kit as claimed in claim 15 wherein said kit further includes a punch driver.

20. A sterile suture anchor and inserter kit as claimed in claim 19 wherein said punch driver comprises a steel cylindrical shaft with a triangular shaped tip.

21. (canceled)
